(19) United States
(12) Patent Application Publication
(10) Pub. No.: US 2015/0352242 A1
(43) Pub. Date: Dec. 10, 2015

(54) AIR PURIFICATION DEVICE

(71) Applicant: SEIWA KOGYO CO., LTD., Yokohama-shi, Kanagawa (JP)

(72) Inventors: Hitoshi Ando, Kanagawa (JP); Nobuki Kuriyano, Kanagawa (JP)

(21) Appl. No.: 14/760,739

(22) PCT Filed: Jan. 23, 2013
(86) PCT No.: PCT/JP2013/051259

§ 371 Date: Jul. 14, 2015

(51) Int. Cl. A61L 9/20; A61L 9/12

(52) U.S. Cl. CPC A61L 9/205; A61L 9/12

(57) ABSTRACT

Provided is an air purification device which can suppress production of side products when purifying air. An air purification device comprises a vent body through which air passes, a photocatalyst filter which is provided inside the vent body, an ultraviolet lamp which applies ultraviolet rays to the photocatalyst filter and an ozone lamp which is provided inside the vent body and generates ozone.

### TECHNICAL FIELD

This invention relates to an air purification device.

### BACKGROUND ART

There are devices which use photocatalyst as well as ultraviolet light and ozone as the devices for purifying air by reacting odorous materials and harmful materials in the air.

Japanese Patent Application Laid-Open Publication No. JP2004-113621 discloses an air purification device in which a photocatalyst body formed of a substrate with a photocatalyst coating film thereon is disposed adjacent to an ultraviolet lamp, an air about to be purified is introduced from an inlet by mans of a fan, the air is purified by the photocatalyst body, thereafter the air which is purified by the photocatalyst is ejected from an outlet, and an ozone generating means and an ozone decomposition means are further provided.

Japanese Patent Application Laid-Open Publication No. JP2000-140087 discloses a sterilization and deodorization device in which air blowing fans and photocatalyst filters of three dimensional structure formed with a plurality of vent pores are disposed so that an ultraviolet lamp is disposed therebetween, the surface of the rotary vane of the air blowing fans is configured to reflect the ultraviolet light, an ozone lamp is juxtaposed to the ultraviolet lamp, and the ultraviolet lamp and the ozone lamp are selectively lighted.

### SUMMARY

However, if the odorous materials and the harmful materials are not sufficiently reacted, side products are generated; the side products can be harmful, i.e. which can be more harmful than the materials contained in the air before the purification. Thus, the problem exists in that the harmful side products are generated after the purification of the air.

This invention aims to provide an air purification device being capable of suppressing the generation of side products during the purification of the air.

In order to achieve the above purpose, an air purification device according to the present invention has: a vent body inside of which air passes through; photocatalyst filters disposed in the vent body; an ultraviolet lamp for irradiating ultraviolet light toward the photocatalyst filter; and an ozone lamp for generating ozone and disposed in the vent body.

In this way, the generation of the side products during the purification of the air can be suppressed.

Preferably, the ozone lamp is disposed in the same level with or in the more upstream side than the ultraviolet lamp with respect to a flowing direction of the air flowing through inside the vent body.

In this way, with comparing to the case which has no constitution of the present invention, the generation of the side products during the purification of the air can be more effectively suppressed.

Preferably, the air purification device has: a first purification area which is formed so that the ozone lamp is sandwiched by the photocatalyst filters with respect to the flowing direction of the air flowing through inside the vent body; and a second purification area which is disposed in a downstream portion of the first purification area and is formed so that the ultraviolet lamp is sandwiched by the photocatalyst filters with respect to the flowing direction of the air flowing through inside the vent body.

In this way, with comparing to the case which has no constitution of the present invention, the generation of the side products during the purification of the air can be more effectively suppressed.

Preferably, the ultraviolet lamps are disposed many more than the number of ozone lamps. In this way, with comparing to the case which has no constitution of the present invention, the generation of the side products during the purification of the air can be more effectively suppressed, and the ejection of ozone to the outside can be also suppressed.

Preferably, the ozone lamp is disposed in approximately the center with respect to the perpendicular direction to the flowing direction of the air flowing through inside the vent body. In this way, the region in which the ozone does not diffuse can be reduced.

According to the air purification device of the present invention, the generation of the side products during the purification of the air can be more effectively suppressed.

### BRIEF DESCRIPTION OF THE DRAWINGS

FIG. 1 shows a schematic view of an air purification device according to one embodiment of the present invention.

FIG. 2 shows a schematic view in the plane along the line II-II of FIG. 1.

FIG. 3 shows a schematic view in the plane along the line III-Ill of FIG. 1.

FIG. 4 shows a measuring result of the change of xylene concentration over time.

### EXEMPLARY EMBODIMENT OF THE INVENTION

The air purification device 10 will be explained.

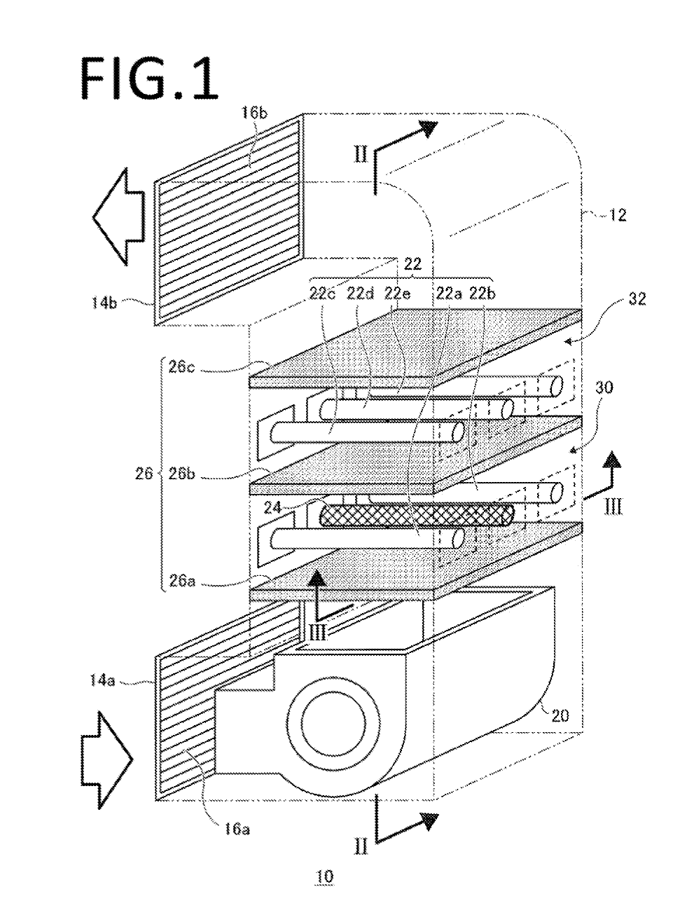

FIG. 1 shows the schematic view of the air purification device 10.

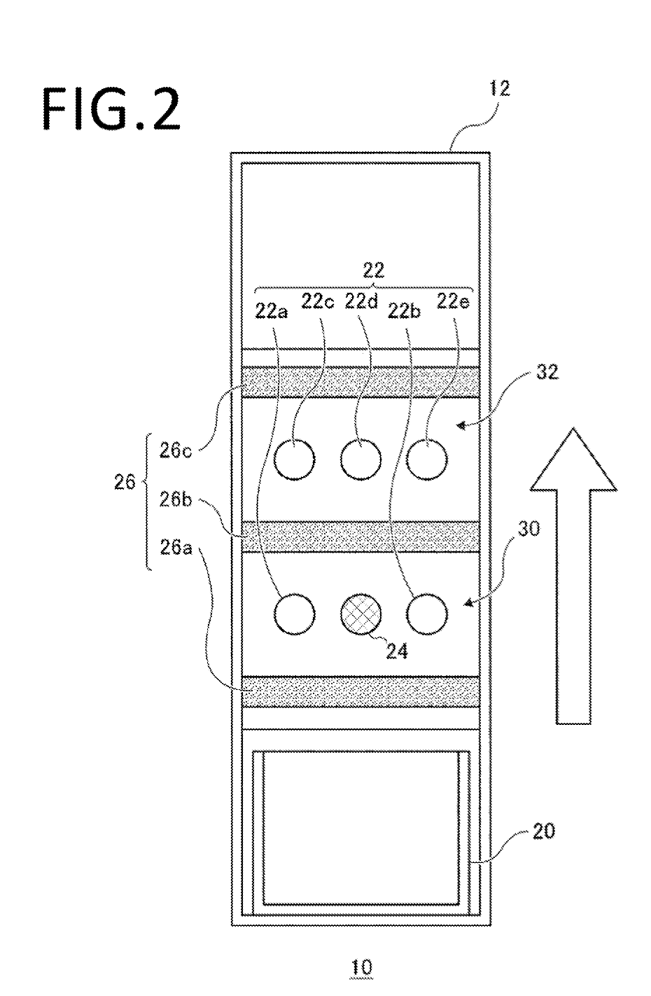

FIG. 2 shows the schematic view in the plane along the line II-II of FIG. 1.

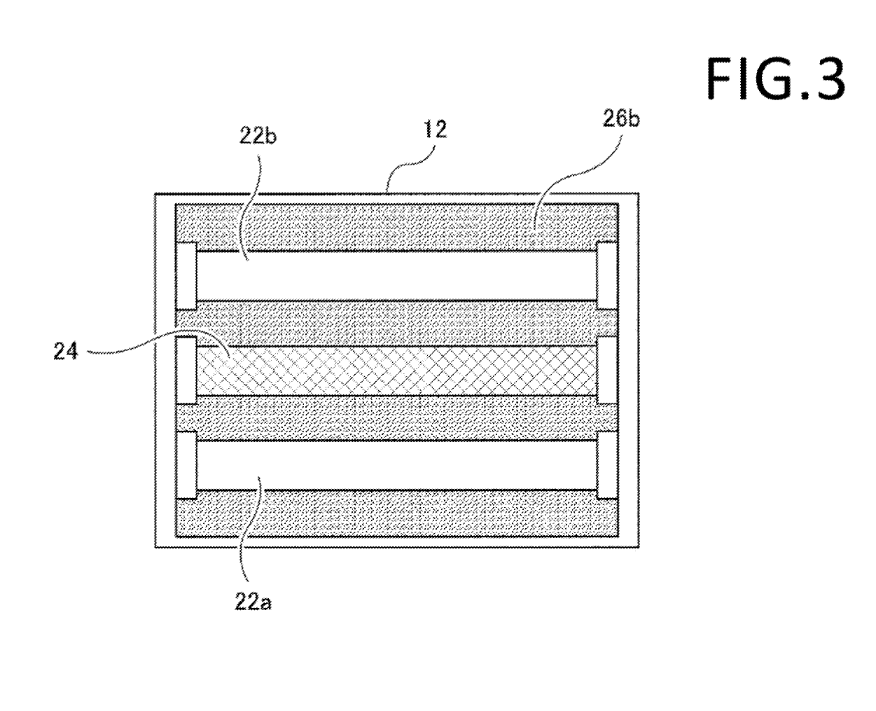

FIG. 3 shows the schematic view in the plane along the line III-III of FIG. 1.

The air purification device 10 has a vent body 12. The vent body 12 is formed with openings 14a, 14b at both sides thereof, and the openings 14a, 14b is provided with filters 16a, 16b, respectively. The vent body 12 is configured so that the air can flow therethrough.

A fan 20, a plurality of, i.e. five in this embodiment, ultraviolet lamps 22, an ozone lamp 24, and a plurality of, i.e. three in this embodiment, photocatalyst filters 26 (they are referred to as photocatalyst filters 26a, 26b, and 26c from the side near the fan 20 in order) are disposed inside the vent body 12.

The fan 20, which is a fan module for example, is disposed adjacent to the opening 14a. The fan 20 draws the air from the opening 14a via the filter 16a, and send the drawn air so as to direct toward the opening 14b passing through the inside of the vent body 12.

The ultraviolet lamps 22 irradiate an ultraviolet light to excite the photocatalyst of the photocatalyst filters 26. The ultraviolet lamps 22 irradiate the ultraviolet light which wavelength is 380 nm or less, more specifically, 351±2 nm or 368*2 nm. Black lights, mercury lamps, and LEDs are used as the ultraviolet lamps 22 for example.

As for the ultraviolet lamps 22, two of which (referred to as ultraviolet lamps 22a, 22b) are disposed between the photocatalyst filter 26a and the photocatalyst filter 26b while three of which (referred to as ultraviolet lamps 22c, 22d, and 22e) are disposed between the photocatalyst filter 26b and the photocatalyst filter 26c.

The ultraviolet lamp 22a and the ultraviolet lamp 22b are disposed in the same level i.e. in line with each other with respect to the flowing direction of the air passing through the inside of the vent body 12 (hereinafter, it can be referred to as “flowing direction of the air”). Further, the ultraviolet lamp 22c, ultraviolet lamp 22d, and the ultraviolet lamp 22e are disposed in the same level i.e. in line with each other with respect to the flowing direction of the air.

The ozone lamp 24 is a lamp generating ozone and irradiating an ultraviolet light which wavelength is 185 nm or less.

The ozone lamp 24 is disposed between the photocatalyst filter 26a and the photocatalyst filter 26b and in approximately the center with respect to the perpendicular direction to the flowing direction of the air. If the ozone lamp 24 is disposed in approximately the center, in comparison to the case in which it is not disposed in the center, the ozone can be easily reached to the entire such as the air which is passing through and the photocatalyst filters 26.

The ozone lamp 24 is disposed in the same level i.e. in line with the ultraviolet lamps 22a, 22b with respect to the flowing direction of the air. That is to say, the ozone lamp 24 is disposed in the same level with the ultraviolet lamps 22a, 22b with respect to the flowing direction of the air, and is disposed in the more upstream side than the ultraviolet lamps 22c, 22d, and 22e. Therefore, the ozone generated by the ozone lamp 24 can be easily reached to a larger extent, thus the region in which the ozone does not diffuse is reduced.

The photocatalyst filters 26 are for reacting and removing odorous materials and harmful materials (hereinafter, they can be referred to as “removable object”). The photocatalyst filters 26 are three dimensional reticulated structures and are formed in flat plate shapes.

In this embodiment, the photocatalyst filters 26 include substrates which are formed of porous ceramic and a photocatalyst layer formed on the surfaces of the substrates and including titanium oxide. A middle buffer film may be formed between the substrate and the photocatalyst layer. The middle buffer film is made of alumina (Al₂O) and is to enhance the adhesiveness of the photocatalyst layer against the substrate.

It is preferred for the substrate to have a high porosity and a large surface area. If the porosity is too small, the resistance against the air flow comes to be large. The larger the surface area is, the better the reaction efficiency in the photocatalyst filter 26 enhances.

Oxide ceramic such as alumina, silica, and cordierite (2MgO, 2Al₂O₃, and 5SiO₂); and non-oxide ceramic such as silicon carbide, and silicone nitride can be employed for the ceramic which constitutes the substrate. The ceramic made of a mixture of the above can also be employed.

As for titanium oxide contained in the photocatalyst layer, an anatase type titanium oxide which exhibits a relatively high activity can be employed, for example. As titanium oxide is irradiated with an ultraviolet light, active species such as OH radicals can be generated and this active species breaks molecular bonds of an organic compound. In this way, the removal object can be decomposed and removed.

The air purification device 10 is formed with a first purification area 30 and a second purification area 32. The first purification area 30 is an area formed so that the ultraviolet lamps 22a, 22b and the ozone lamp 24 are sandwiched by the photocatalyst filters 26a, 26b. The second purification area 32 is disposed in the downstream portion of the first purification area 30 with respect to the flowing direction of the air and is an area formed so that the ultraviolet lamps 22c, 22d, and 22e are sandwiched by the photocatalyst filters 26b, 26c.

An ozone removing device for removing ozone may be disposed between the photocatalyst filter 26c and the opening 14b. By disposing the ozone removing device, the ozone generated with the ozone lamp 24 can be restricted to eject outward of the air purification device 10.

Next, some specific details of the removing capability for removing the removal object will be explained with making use of the following Example 1 and Comparative Example 1.

### Example 1

In Example 1, the air purification device in which five ultraviolet lamps 22 and one ozone lamp 24 are disposed are used. As for the photocatalyst filters 26, width of 260 mm×length of 275 mm×thickness of 20 mm was used.

The photocatalyst filter 26 was manufactured as follows:

A ceramic which main component is SiC (about 67%) —Al₂O₃ (about 21%) —SiO₂ (about 12%) was prepared for the substrate. A gel liquid containing titanium dioxide was prepared as the coating material for forming the photocatalyst layer.

Then, the substrate was immersed into the coating material so that the coating material adheres onto the whole surface of the substrate. Thereafter, the resultant substrate was dried at a temperature of 1000° C. or low. The immersion and the drying was repeatedly implemented so that the photocatalyst layer would be a predetermined thickness. Then, the resultant material was baked at a temperature from 1300° C. to 1500° C.

### Comparative Example 1

The air purification device in Comparative Example 1 was the same constitution as Example 1 except for replacing the ozone lamp 24 with the ultraviolet lamp 22. That is to say, in the air purification device of Comparative Example 1, the ozone lamp 24 was not used but six ultraviolet lamps 22 are disposed.

### [Measurement for Air Purification Capability]

The air purification capabilities with regard to the air purification devices of Example 1 and Comparative Example 1 were measured. The measurement for the air purification capability was implemented in compliance with the standards of the testing methodology for deodorizing performance of “domestic air cleaner”, recited in JEM1467, Japan Electrical Manufacture's Association.

Specifically, the air purification device was disposed in an air tight container of volume 1 m3, which is made of acrylic resin, and was sealed up, thereafter xylene as the removal object was diffused inside the air tight container with permeating into a filter paper. After the xylene concentration (initial concentration) in the air tight container was stabilized, the air purification device was activated for 120 minutes. Then the xylene concentration was sequentially measured.

The initial concentration was controlled to be 10 ppm.

The measurement of the concentration was implemented by making use of Photoacoustic Multi-gas Monitor (Model 1312 produced by INNOVA).

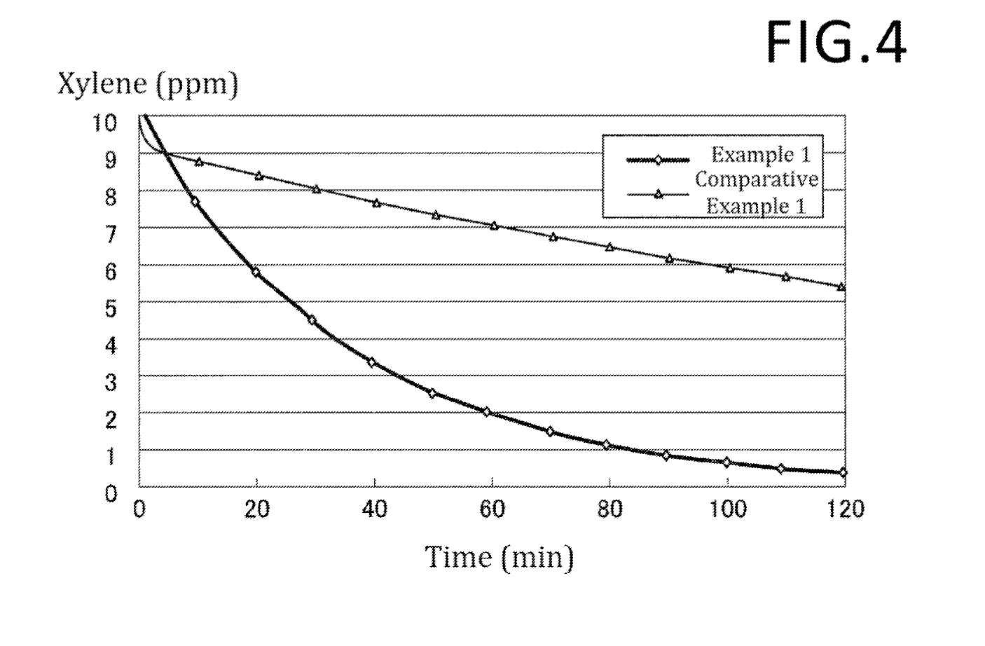

FIG. 4 shows a measurement result of the change of xylene concentration over time.

In Comparative Example 1, the xylene concentration after 120 minutes from the start was approximately 6 ppm. On the other hand, in Example 1, the xylene concentration after 20 minutes from the start was approximately 6 ppm and the xylene concentration after 120 minutes from the start was 0.5 ppm or less. It is understood in Example 1 that, in comparison with Comparative Example 1, the xylene concentration is rapidly decreased in a curved state over the time. In Example 1, the xylene concentration after 120 minutes from the start is also decreased in comparison with Comparative Example 1. That is to say, the device of Example 1 has a high xylene removing capability in comparison to the Comparative Example 1.

### [Identification of Side Products]

The side products afterward the removal of the removal object was identified with regard to the air purification devices of both Example 1 and Comparative Example 1. The identification of the side products was implemented in compliance with “Chapter 2, A Measurement Method for Volatile Organic Compound Content in the Atmosphere such as Benzene” of the Manual for Harmful Atmospheric Pollutant Measurement Method (revised in October, 2008 by Ministry of Environment).

Specifically, after the air purification capability was measured as described above (i.e. the air purification device was activated for 120 minutes), the gas inside the sealed container was sampled by means of a pump and was adsorbed to a GASTEC (spherical activated carbon sampling tube 258). Then, the side products were analyzed by making use of a gas chromatograph mass spectrometer (GCMS-QP2010 produced by SHIMADZU CORPORATION, Column: InterCap1 (0.25 mm×60 m×25 μm)).

The sampling condition of the gas from the sealed container was: 500 mL/min for 10 minutes (total of SL).

The table 1 shows the identification result of the side products.

TABLE 1

| | Ozone | Xylene | Toluene | Benzaldehyde | Cumene |
|---|---|---|---|---|---|
| | lamp | (ppm) | (ppm) | (ppm) | (ppm) |
| Comparative | presence | 5.45 | 0.29 | 0.22 | n.d. |
| Example 1 | | | | | |
| Example 1 | absence | 0.38 | 0.01 | 0.003 | n.d. |

In Comparative Example 1, the xylene concentration after the 120 minutes of activation was 5.45 ppm, a half or more xylene which was diffused at first was still remained without being removed. In Comparative Example 1, toluene was produced at 0.29 ppm, and benzaldehyde was produced at 0.22 ppm.

In contrast, in Example 1, the xylene concentration after the 120 minutes of activation was 0.38 ppm, thus the most of xylene was removed. In Example 1, toluene was produced at 0.001 ppm, and benzaldehyde was produced at 0.003 ppm, thus the generating amount of the side products was small in comparison with Comparative Example 1.

Cumene was not recognized to be produced in Comparative Example 1 and Example 1.

In general, aromatics (especially aromatic rings) such as toluene, xylene, and benzaldehyde are materials which are hard to be broken during the purification of the air. The aromatics, even if they are reacted though, the reaction can be easily stopped at the step of the oxide i.e. at the step of oxide of the aromatics being formed.

For example, when the xylene contained in the air is broken, toluene and benzaldehyde can be produced a lot as the side products due to the broken process. Even if the removal object substance is broken, though, if these side products are still produced, it can be said that the air is not purified or detoxificated. In Comparative Example 1, xylene was broken in a certain extent, however, the side products were produced in relatively large amount. In contrast, in Example 1, toluene and benzaldehyde were restricted to be produced in addition to the most of xylene being removed.

The reason for high removing capability and for restricting the production of the side products in Example 1 is estimated as follows:

The removal object is removed by: the ultraviolet light irradiated from the ultraviolet lamps 22; the photocatalyst of the photocatalyst filters 26 excited by the ultraviolet light; the ozone generated by the ozone lamp 24; and their effect. In addition to the above, in Example 1, the photocatalyst is considered to be excited by the ultraviolet light irradiated from the ozone lamp 24. Due to the above, the activity of the photocatalyst is enhanced. Further, by making ozone exist upon the excited photocatalyst, it is considered that more prominent effect than the effect of the case in which these elements are only combined can be achieved.

Further, in Example 1, the reaction pathway during the removal of the xylene is considered to be different from the one in the case such as Comparative Example 1 in which other device is used.

In Comparative Example 1, it is considered that one of two methyl groups belonging to xylene is separated thereby the toluene is produced, the methyl group of the toluene is oxidized to produce the benzaldehyde, thus the xylene is removed. In contrast, in Example 1, it is considered that the aromatic ring of the xylene is decomposed to produce a chain substance. That is to say, in Example 1, it is considered that the xylene is removed without producing the toluene and the benzaldehyde.

In this way, according to the air purification device 10 of the embodiment, removing capability for the removal object is enhanced in comparing with the case which has no present constitution. Especially, an amount of aromatics existed afterward the air purification is decreased. Further, ozone is a material which is harmful to human body. With this view point, it is preferred that quantity of generation of ozone is small. According to the air purification device 10, since the photocatalytic reaction by the photocatalyst filters 26 is simultaneously used, the generation quantity of ozone is restricted while the air is effectively purified.

In the air purification device 10, it is preferred that the ultraviolet lamps 22 are disposed many more than the number of ozone lamps 24. Taking the size of the device and the distance toward the photocatalyst filters 26 into the account, the numbers of the ultraviolet lamps 22 and the ozone lamps 24 which can be disposed in the device are limited. Therefore, disposing many ozone lamps 24 will likely lead to decrease the number of the ultraviolet lamps 22 being able to be disposed. If the ultraviolet lamps 22 are decreased, the photocatalytic reaction of the photocatalyst filters 26 comes to be less effective. Further, if the number of the ozone lamps 24 is increased, since the generation quantity of ozone is increased, the ozone comes to be easily ejected to outside of the air purification device 10.

In the above embodiment, it is explained that the constitution has the five ultraviolet lamps 22, and the one ozone lamp 24, however, these numbers of the ultraviolet lamps 22 and the ozone lamps 24 are arbitrarily selected.

Further, the ultraviolet lamps 22 and the ozone lamps 24 can be twin pipes.

1. An air purification device, comprising:
a vent body inside of which air passes through;
photocatalyst filters disposed in the vent body;
an ultraviolet lamp for irradiating ultraviolet light toward the photocatalyst filters;
an ozone lamp for generating ozone and disposed in the vent body.

2. The air purification device according to claim 1, wherein the ozone lamp is disposed in the same level with or in the more upstream side than the ultraviolet lamp with respect to a flowing direction of the air flowing through inside the vent body.

3. The air purification device according to claim 1, wherein
the air purification device further includes: a first purification area which is formed so that the ozone lamp is sandwiched by the photocatalyst filters with respect to a flowing direction of the air flowing through inside the vent body;
a second purification area which is disposed in a downstream portion of the first purification area and is formed so that the ultraviolet lamp is sandwiched by the photocatalyst filters with respect to the flowing direction of the air flowing through inside the vent body.

4. The air purification device according to claim 1, wherein the ultraviolet lamps are disposed many more than the number of ozone lamps.

5. The air purification device according to claim 1, wherein the ozone lamp is disposed in approximately the center with respect to the perpendicular direction to a flowing direction of the air flowing through inside the vent body.
